(19) United States
(12) Patent Application Publication
(10) Pub. No.: US 2022/0392567 A1
(43) Pub. Date: Dec. 8, 2022

(54) MODELLING FRAMEWORK FOR EMBEDDING-BASED PREDICTIONS FOR COMPOUND-VIRAL PROTEIN ACTIVITY

(71) Applicant: Qatar Foundation for Education, Science and Community Development, Doha (QA)

(72) Inventors: Sanjay Chawla, Doha (QA); Ehsan Ullah, Doha (QA); Raghvendra Mall, Doha (QA); Hossam Almeer, Doha (QA); Abdurrahman Elbasir, Doha (QA)

(21) Appl. No.: 17/804,408

(22) Filed: May 27, 2022

Related U.S. Application Data
(60) Provisional application No. 63/193,845, filed on May 27, 2021.

(51) Int. Cl. G16B 15/30; G16B 40/20; A61K 31/438; G06N 20/20

(52) U.S. Cl. CPC G16B 15/30; G16B 40/20; A61K 31/438; G06N 20/20

(57) ABSTRACT

A global effort is underway to identify compounds to treat emerging virus infections, such as COVID-19. Since de novo compound design is an extremely long, time-consuming, and expensive process, efforts are underway to discover existing compounds that can be repurposed for COVID-19 and new viral diseases. The present invention discloses a machine learning representation framework that uses deep learning-induced vector embeddings of compounds and viral proteins as features to predict compound-viral protein activity. The prediction model uses a consensus framework to rank approved compounds against viral proteins of interest.

### CROSS-REFERENCE TO RELATED APPLICATIONS

The present disclosure claims the benefit of U.S. Provisional Patent Application 63/193,845 titled “A MODELLING FRAMEWORK FOR EMBEDDING-BASED PREDICTIONS FOR COMPOUND-VIRAL PROTEIN ACTIVITY”, filed on May 27, 2021, and which is incorporated by reference herein in its entirety.

### BACKGROUND

The outbreak of the novel coronavirus disease, COVID-19, caused by the new coronavirus 2019-nCoV that is now officially designated as severe acute respiratory syndrome-related coronavirus SARS-CoV-2, represents a pandemic threat to global public health. Since the outbreak of COVID-19, this new disease and its causative virus have drawn major global attention. Scientists and physicians have been trying to understand this new emergent disease and its epidemiology in an effort to uncover possible treatment regimens, discover effective therapeutic agents, and develop vaccines.

There is an immediate need for effective treatment to contain the spread of this pandemic. Based on the time and resources required to develop new compounds to treat COVID-19 and emerging viral diseases, it is not feasible to rely completely on the traditional process of compound discovery, which takes an average 15 years and costs $2-3 billion to bring a new compound to market. A more pragmatic approach would be to perform drug repurposing, more specifically, accurately identify a set of candidate compounds which can exhibit high activity against viral proteins and potentially inhibit them using novel in-silico techniques.

Identification of targets is important for identifying drugs with high target specificity and/or uncovering existing drugs that could be repurposed to treat SARS-CoV-2 infection. Since SARS-CoV-2 is a newly discovered pathogen, no specific drugs have been identified or are currently available. A genomic sequence information coupled with protein structure modeling could accelerate the identification of existing drugs with therapeutic potential for COVID-19.

Accordingly, there is a need for a research tool that can accurately identify a set of candidate compounds which can exhibit high activity against viral proteins and potentially inhibit them.

### SUMMARY

The present disclosure provides a new and innovative method for predicting activity value for compound-viral protein interactions. The method uses data-drive machine learning models based on a simplistic representation of compounds (simplified molecular-input line-entry systems (SMILES) strings or Morgan Fingerprints) and viral protein sequence (amino acid (AA) sequence) to accurately predict activity value for compound-viral protein interactions. The method may further use two-dimensional images of compounds and physio-chemical and structural properties of proteins to strengthen the model. An aim of the provided method is to accurately identify a set of candidate compounds which can exhibit high activity against viral proteins and potentially inhibit them using novel in-silico techniques.

The present disclosure provides a method for predicting an activity value for compound-viral protein interaction, employing a consensus framework of in-silico embedding-based modeling techniques, which use different combinations of representations for compounds and viral proteins including: Morgan Fingerprints (MFP) as chemoinformatic descriptors of compounds+a convolutional neural network (CNN) autoencoder based vector representation for viral protein sequence; a teacher forcing−long short term memory neural network (TFLSTM) autoencoder based vector representation for compounds+CNN autoencoder based vector representation for viral proteins; canonical SMILES based sequential representation of compounds+Primary structure (linear chain of amino acid) based sequential representation of viral proteins.

The present disclosure encompasses several advantages over existing models predicting the compounds-protein binding affinity, such as using already collected information from other viruses to infer virus-specific compound activity when new viruses emerge to reduce costs and save time. Additionally, unlike the most commonly used AI prediction methods, the present disclosure avoids training deep learning models on human protein sequences (e.g., kinases, nuclear receptors, G-protein-coupled receptors) that are significantly different from viral protein sequences. Finally, the present disclosure provides the ability to collect information about the primary structure (e.g., linear chain of amino acids) for proteins associated with viruses, instead of using molecular docketing that requires high-quality three-dimensional crystal structures of the protein of interest as well as annotation information about the presence of active sites. Therefore, the present disclosure provides the prediction model for compound-viral protein activity that is cost-effective and time-efficient.

### BRIEF DESCRIPTION OF THE DRAWINGS

FIG. 1 is a block diagram of a data gathering framework, according to embodiments of the present disclosure.

FIG. 2 is a block diagram of regression models used in a consensus framework, according to embodiments of the present disclosure.

FIG. 3 is a flowchart of a method for applying the consensus framework, according to embodiments of the present disclosure.

### DETAILED DESCRIPTION

The present disclosure provides a method for predicting an activity value for compound-viral protein interaction by employing a consensus framework of in-silico embedding-based modeling techniques. The proposed method uses a machine learning representation framework that uses deep learning-induced vector embeddings of compounds and viral proteins as features to predict compound-viral protein activity. The prediction model, in turn, uses a consensus framework to rank approved compounds against viral proteins of interest. This prediction model allows the cost and time-efficient identification of compounds for the treatment of emerging viral infection, such as COVID-19.

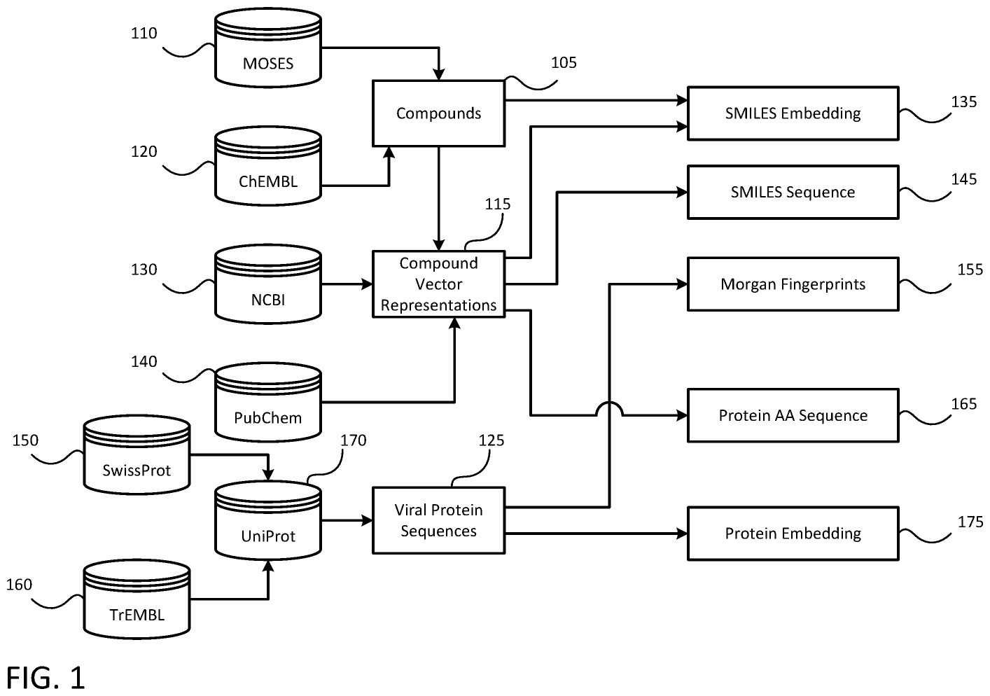

In FIG. 1, information about compounds, viral protein sequences, and compound-viral protein interactions (activity values) is collected from resources such as MOSES 110, ChEMBL 120, UniProt 170, PubChem 140, and NCBI 130 databases, to build in-silico embedding-based compound-viral protein activity predictors.

In an embodiment, for data collection of compounds 105, the dataset representing ≈2.5 million simplified molecular-input line-entry systems (SMILES) for compounds is collected. This dataset is then filtered to remove salts and stereochemical information. The final compound set S consists of approximately 2.5 million canonical SMILES sequences 145 for small molecules. To train the traditional supervised machine learning (ML) algorithms, the set S is used to train a TF-LSTM based autoencoder, which generates a low dimensional vector representation (LS_{c}) for each compound (e.g., compound vector representations 115). In addition, traditional cheminformatic descriptors such as Morgan Fingerprints (MFP) 155 derived from compound structures as an alternative vector representation for each compound are used.

In an embodiment, for data collection of viral proteins, the viral protein sequences 125 available in UniProt 170, comprising a total of approximately 2.7 million protein sequences, are downloaded. Among these, approximately ten thousand are deposited in SwissProt 150 (which are manually crated and functionally annotated), whereas the remaining protein sequences are obtained from TrEBML 160 and are not well-curated. The viral protein sequences 125 are filtered to keep sequences with L≤2000, resulting in a set V of approximately ≈99% of all viral proteins available in UniProt 170. The set V is used to train a CNN based autoencoder which then generates the required low dimensional representation (LSᵥ for each viral protein sequence.

In the data gathering shown in FIG. 1, ≈2.5 million SMILES representations of compounds were collected from MOSES 110 and ChEMBL 120 databases. These representations are used to learn a SMILES embedding 135 representation (numeric vector representation) via a TF-LSTM autoencoder model. In addition, ≈2.5 million viral protein amino acid (AA) sequences 165 were collected from Uniprot 170 database. These are passed through a CNN autoencoder to learn viral protein embedding 175 representation (numeric vector representation). Compound-viral protein activities were collected, curated, and assimilated from resources such as NCBI 130, PubChem 140, and ChEMBL 120 to build a dataset (D). The corresponding bioactivities in these samples are transformed into a standardized pChEMBL value measured in nanomolar (nM) concentration and are used to build downstream regression models. In various embodiments, values presented in PubChem standards are converted to a standardized pChEMBL value where pChEMBL=−log₁₀(Activity_{pubchem})+6, where Activity_{Pubchem} corresponds to one of IC₅₀, EC₅₀, AC₅₀, Kᵢ, K_{d}, Potency.

The compound-viral protein activities compares viral proteins and small molecules to target these viral proteins, to identify the bioactivities between the compounds and viral activities, The bioactivities can include measurements such as IC₅₀, EC₅₀, AC₅₀, Kᵢ, K_{d}, Potency, and other standard potency measures derived from dose-response assays at different concentration designed to measure activation, inhibition of targets, and pathways pharmacological significance.

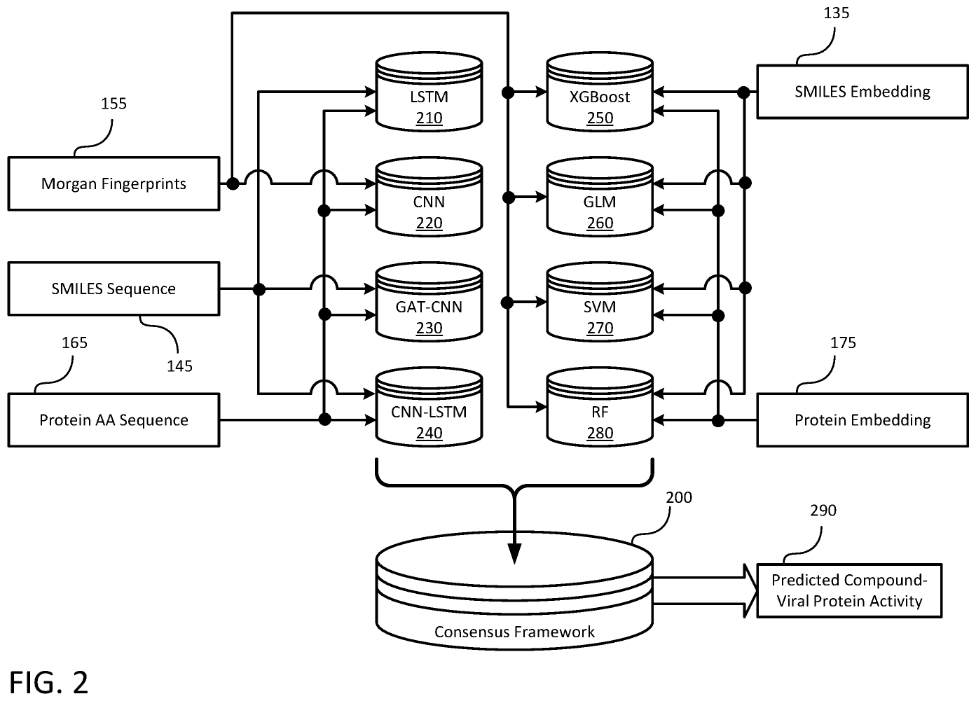

FIG. 2 is a block diagram of regression models used in a consensus framework 200 to identify a predicted compound-vital protein activity 290, according to embodiments of the present disclosure. These regression models are developed using various machine learning (ML) techniques that take advantage of different representations of compounds and viral proteins for in-silico compound-viral protein activity prediction. A consensus of the top N (e.g., N=5) predictors is derived based on their performance with respect to a predefined number P (e.g., P=4) of evaluation metrics on the test set. According, a plurality of different models are constructed using a plurality of different ML architectures, and are evaluated against P metrics to identify the N top performing models, which are then used (ignoring the low performing models) to form a consensus on the predicted compound-viral protein activity. In various examples, the P evaluation metrics can include any of: the mean absolute error, mean squared error, Pearson correlation R, and the coefficient of determination.

These models may use different inputs, and reach conclusions in different ways, and the consensus between the models ensures accuracy in the prediction generated under the consensus framework 200. The compound-viral protein activity prediction for each model can be posed as a regression task to learn a mapping function g that receives joint compound and viral protein representations (x_{c}, xᵥ) and outputs the activity value y_{cv} for that pair. With l defined as the model-specific loss function, the regression tasks reduce to estimate the parameters w, which minimizes Formula 1.

min_{w}Σ_{c,v}l(y_{cv},g(x_{c}xᵥ,w) Formula 1

The mapping function g is a ML method including a Generalize Linear Model (GLM) 260, XGBoost 250 model, Support Vector Machine (SVM) 270 model, and l is the squared loss function. In these models, x_{c} may be passed to a TG-LSTM or Morgan fingerprint generator, and xᵥ is passed to a CNN to generate a numeric vector representation LS_{c} (for compounds) and LSᵥ (for viral proteins), which are used in the ML models to estimate activity values according to Formula 2.

ŷ_{cv}=g(LS_{c},LSᵥ,w) Formula 2

In additional examples, the LSTM 210, GAT-CNN 230, and CNN-LSTM 240 models may receive inputs of the SMILES sequences 145 and the protein AA sequences 165, while the CNN 220 receives inputs from the protein AA sequences 165 and Morgan Fingerprints 155, while the XGBoost 250, GLM 260, SVM 270, and RF 280 models receive inputs from the Morgan fingerprints 155, SMILES embedding 135, and the protein embeddings 175 gathered, filtered, and processed as explained in relation to FIG. 1. Each of these models predicts an individual compp

In an embodiment, four end-to-end deep learning models are built for the regression problem where the mapping function g were CNN, LSTM, CNN-LSTM, and GAT-CNN. These models directly work on the compound (x_{c}) and viral protein (xᵥ) representations, unlike traditional ML techniques.

The LSTM model 210 includes two LSTM encoders. It includes an LSTM encoder based on the compound representation (x_{c}) and another one based on the viral protein representation (xᵥ). The compound LSTM encoder generates the hidden state vector (h_{c}) while the viral protein encoder generates the hidden state vector (hᵥ). The two hidden vectors are then concatenated together (h). Multiple feed-forward layers are then layered on top of h which is connected to the output unit representing the activity value. The LSTM encoders not only capture short but long term dependencies as well, due to the availability of memory units, based on SMILES strings and viral protein sequences and the feed-forward layers encapsulate the co-occurrence of such patterns driving the activity value to be high or low for a given compound-viral protein combination.

The CNN 220 model comprises two CNN encoders. For the compound and protein CNN encoders, each of the compound (x_{c}) and viral protein (xᵥ) representation is passed through an embedding layer (e(·) to generate compound embedding matrix and viral protein embedding matrix respectively. A single convolutional layer with multiple filter sizes, k∈K={3,6,9,12}, is applied on top of the embedding matrix followed by a max-pooling operation to generate hidden state vector for small molecules as well as viral protein sequences. The hidden state vector h_{c} for compounds and by for viral protein sequences are then concatenated together (h) and are considered as the output of the CNN encoders. Multiple feed-forward layers are then layered on top of h which are ultimately connected to the output unit corresponding to the activity value. The CNN encoders can capture contiguous sequences in the SMILES representations and k-mers in viral protein sequence, whereas the feed-forward layers capture the co-occurrence of such patterns that drive the activity value to be either high or low based on our training set Dₜᵣₐᵢₙ. Non-linear activations are used at every layer, and the model architecture w.r.t. hyper-parameters, such as filter sizes, learning rate, etc., are optimized.

The CNN-LSTM 240 model is a combination of the CNN 220 and the LSTM 210 models. By combining the CNN 220 and LSTM 210 models, the CNN-LSTM 240 model can capture spatially contiguous and well as long-term dependencies in the SMILES strings and viral protein sequences. The output of each encoder is concatenated together to generate hidden representation h, which is passed to multiple feed-forward layers and is ultimately connected to the output layer consisting of one unit for the activity value.

Graph Attention Networks-Convolutional Neural Networks (GAT-CNN) 230 model is composed of two parts, graph attention networks and convolutional neural networks. For a given compound, the compound structure can be presented as a graph consisting of the atoms (nodes) in the compound and connected by edges if a bond exists between a pair of atoms. In various embodiments, to convert a compound structure to the form of graph representations, the RDKit package taking SMILES strings may be used. Furthermore, RDKit can be used to extract different atom features such as atom's degree, the total number of hydrogen, the number of hydrogen with the number of bonded neighbors, atom status as aromatic or not, the implicit value of atoms, and atom symbol. These features can be used as node properties for atoms. In various embodiments, the atoms can include 78 such features from the SMILES strings. Given the graph-based representation of a compound molecule (x_{c}) along with the extracted node features, the GAT portion of the GAT-CNN 230 model learns an embedding representation for a compound encapsulating the topological information available in the graph of each compound. The second component of the GAT-CNN 230 architecture is a CNN which take protein AA sequences as an input. This component is composed of the embedding layer and multiple convolutional layers. At each convolutional layer, a non-linear activation function is applied and is followed by a max-pooling operator. The CNN portion learns protein embedding (hᵥ) and concatenates it with the SMILES embedding (10 generated by GAT portion to produce h, which is then passed to feed-forward layers. The output layer provides the value corresponding to the compound activity.

The consensus framework 200 averages the output activity estimates from the N top performing models to arrive at a consensus value for the predicted compound-viral protein activity 290 to learn from different combinations of non-linear patterns from diverse representations of the input data.

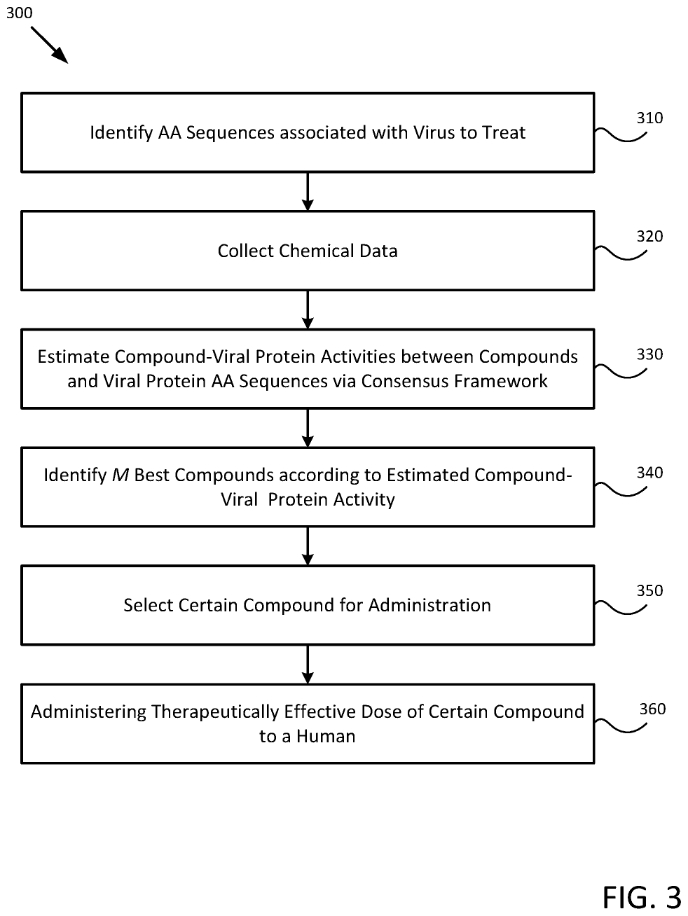

FIG. 3 is a flowchart of a method 300 for applying the consensus framework, according to embodiments of the present disclosure. Method 300 begins with block 310, where a user identifies the Amino Acid (AA) sequences associated with a virus to treat in a human. For example, when treating SARS-COV-2, the user may identify PL-Pro, 3CL-Pro, and the spike proteins as the AA sequences of interest, although in other examples for different viruses, different AA sequences may be selected.

At block 320, a consensus framework collects chemical data for a plurality of compounds to examine for administration to a human to treat the virus and the identified proteins/AA sequences of the virus to treat in the human. In some examples, the consensus model collects the chemical data from a variety of different sources, and standardizes the chemical representations to provide a set of two-dimensional vector embeddings for various candidate compounds at various dosages and the virus AA sequences. In some examples, the chemical data include simplified molecular-input line-entry system (SMILES) representations of a plurality of compounds and viral protein representations for the viral protein amino acid sequences.

At block 330, the consensus framework estimates the compound-viral protein activities between each compound of the plurality of compounds and the viral protein amino acid sequences according to a plurality of machine learning models. In various examples, the consensus framework includes a plurality of different ML models constructed according to a corresponding plurality of different ML architectures, including: a Generalized Linear Model (GLM), Random Forests (RF), XGBoost, Support Vector Machines (SVM), Convolutional neural network (CNN), Long Short Term Memory (LSTM), CNN-LSTM, and Graph Attention Network (GAT)-CNN.

The consensus framework evaluates the outputs of each of the ML models to select the N top performing models to arrive at a consensus value for the predicted compound-viral protein activity by averaging the outputs of those N models. The consensus framework identifies the N top performing models from among the plurality of models based on their performance with respect to a predefined number P (e.g., P=4) of evaluation metrics on the test set, which can include the mean absolute error, mean squared error, Pearson correlation R, and the coefficient of determination for each model.

At block 340, the consensus model identifies the M best compounds according to the estimated compound-viral protein activities. When evaluating a plurality of proteins, the M best compounds may be evaluated based on a threshold value for any one of the proteins or a composite value of two or more proteins relative to each compound. In some examples, the candidate compounds may be evaluated at different doses, where the models evaluate the compounds at different concentrations or dosages, which may be used to identify a therapeutically effective dose when the compound is used in vivo after being evaluated in silico.

For example, when evaluating the PL-Pro, 3CL-Pro, and Spike Protein of SARS-COV-2, the consensus framework may output the M=47 best compounds according to Table 1, wherein the Predicted pChEMBL is the output of the consensus model and the binding energy (in Kcal/mol) is obtained via molecular docking experiment. Table 1 includes examples produced with high activities (e.g., predicted pChEMBL values), and may be further refined to select certain compounds having low binding energies, that are not already being examined or used to treat the virus in question, or that are available for use in trials or in human treatment (e.g., for other conditions).

TABLE 1

| | PL-Pro | | 3CL-Pro | | Spike Protein | |
|---|---|---|---|---|---|---|
| | Predicted | Binding | Predicted | Binding | Predicted | Binding |
| Compound | pChEMBL | Energy | pChEMBL | Energy | pChEMBL | Binding |
| Lopinavir⁺ | 7.777 | −6.3 | 7.851 | −8.7 | 8.226 | −5.0 |
| Ritonavir⁺ | 7.562 | −6.4 | 7.777 | −7.7 | 7.845 | −5.5 |
| Palinavir⁺ | 7.416 | −6.4 | 7.48 | −7.2 | 7.699 | −6 |
| Simeprevir⁺ | 7.646 | −5.6 | 7.476 | −6.1 | 8.206 | −6.2 |
| Cabotegravir⁺ | 7.194 | −7.1 | 6.951 | −9.5 | 7.002 | −6.8 |
| L-870812⁺ | 6.937 | −7.1 | 6.895 | −8.9 | 6.68 | −7.2 |
| MK-4965⁺ | 7.319 | −7.5 | 6.893 | −9.6 | 7.302 | −7.1 |
| Tipranavir⁺ | 6.634 | −7.4 | 6.83 | −8.3 | 6.794 | −6.6 |
| Zanamivir⁺ | 6.798 | −5.7 | 6.801 | −5.9 | 6.748 | −5.9 |
| BMS-707035⁺ | 6.938 | −7.2 | 6.766 | −8.8 | 6.511 | −6.6 |
| GSK-364735⁺ | 7.086 | −6.4 | 6.745 | −9.6 | 6.552 | −7 |
| Paritaprevir⁺ | 6.751 | −6.8 | 6.571 | 5.6 | 7.443 | −6.2 |
| Filociclovir⁺ | 6.542 | −5.7 | 6.463 | −7.1 | 6.647 | −6.2 |
| TMC-647055⁺ | 6.717 | −5.8 | 6.459 | 11.7 | 6.539 | −5.5 |
| Elvitegravir⁺ | 6.462 | −6.8 | 6.402 | −8 | 6.236 | −5.7 |
| Dapivirine⁺ | 6.584 | −6.7 | 6.385 | −8.7 | 6.32 | −6.4 |
| PLX-8394* | 6.208 | −9.1 | 6.358 | −9.4 | 6.494 | −7.2 |
| Triciribine PO₃ * | 6.385 | −6.7 | 6.354 | −8.1 | 6.314 | −6.5 |
| Zibovudine⁺ | 5.966 | −5.7 | 6.279 | −7.4 | 6.264 | −5.6 |
| API-2*^{/+} | 5.964 | −6.7 | 6.175 | −8.3 | 6.208 | −5.7 |
| Fluorouracil⁺ | 5.965 | −4.5 | 6.157 | −5.2 | 6.353 | −4.6 |
| Gossypol* | 6.029 | −5.7 | 6.11 | −4.2 | 6.069 | −6 |
| LM 565⁻ | 6.137 | 8.7 | 6.094 | 72.4 | 6.461 | −2.9 |
| PF-03814735* | 6.051 | −7.6 | 6.091 | −8.2 | 6.126 | −7.1 |
| Barasertib* | 6.006 | −8 | 6.087 | −8.3 | 6.171 | −6.8 |
| Edoxudine⁺ | 5.925 | −5.9 | 6.075 | −7.6 | 6.246 | −5.6 |
| Cefozopran⁻ | 5.884 | −7 | 6.049 | −8.1 | 6.255 | −6 |
| Entrectinib* | 6.231 | −6.8 | 6.039 | −9.3 | 6.023 | −7 |
| Clemizol* | 6.085 | −6.2 | 6.015 | −8 | 6.105 | −6 |
| VBY-825⁺ | 6.112 | −6 | 6.006 | −8 | 6.07 | −4.7 |
| R-763* | 6.158 | −6.6 | 6.002 | −7.8 | 6.26 | −6.7 |
| Bietaserpine © | 6.054 | −6.1 | 5.994 | −2.1 | 6.323 | −4.9 |
| ACT-077825 © | 5.916 | −6.9 | 5.973 | −6.7 | 6.223 | −4.8 |
| MP-412* | 6.069 | −6.6 | 5.971 | −9 | 6.243 | −5.6 |
| Remdesivir⁺ | 5.907 | −6.2 | 5.964 | −8 | 6.37 | −6.4 |
| ABT-263* | 6.005 | −4.2 | 5.925 | 1.9 | 6.211 | −5.6 |
| BMS-903452 © | 5.929 | −6.9 | 5.913 | −7.8 | 6.174 | −6.3 |
| Brilacidin © | 6.016 | −5.7 | 5.913 | −2.2 | 6.266 | −5.2 |
| Taselisib* | 5.934 | −7 | 5.906 | −8.6 | 6.142 | −7.1 |
| Goxalapladib © | 5.982 | −6.9 | 5.905 | −6.6 | 6.27 | −5.1 |
| HKI-357* | 6.009 | −6.8 | 5.884 | −8.7 | 6.143 | −6.2 |
| Sitravatinib* | 5.895 | −6.3 | 5.879 | −8.2 | 6.069 | −7 |
| Rifabutin⁻ | 5.904 | −9.4 | 5.878 | −12.3 | 6.136 | −12.1 |
| Omadacycline⁻ | 6.002 | −6.1 | 5.865 | −2.6 | 6.251 | −5.3 |
| Cefpiramide⁻ | 5.883 | −6.8 | 5.851 | −8.3 | 6.179 | −5.9 |
| VCH-286 © | 5.88 | −6.6 | 5.847 | −8.1 | 6.028 | −4.6 |
| BMS-754807* | 5.915 | −6.6 | 5.833 | −8.3 | 6.095 | −7.1 |

At block 350, a user (or the consensus framework) selects a certain compound for administration or further clinical trials to treat the virus in a human. For example, Rifabutin may be selected for further trials or to treat SARS-COV-2 based on having a consistently low binding energy across the three proteins analyzed according to the examples shown in Table 1. Additionally or alternatively, Rifabutin may be selected for further trials or to treat SARS-COV-2 based on having a lowest binding energy for any of the individual proteins under analysis. In another example, LM 565 may be selected for further trials or for treating SARS-COV-2 based on having very high values for PL-Pro and 3CL-Pro, potentially indicating that LM 565 is a false positive for inclusion in the list displayed in Table 1.

At block 360, a user administers a therapeutically effective dose of the certain compound selected per block to a human as part of a clinical trial of the compound in treating the virus, or to directly treat the virus in question. Data from the in vivo use of the compound to treat the virus may be collected and fed back into the data sets used by the consensus framework, and may be used a training data for the various ML models used by the consensus model to improve the efficacy and accuracy of the ML models in identifying different compounds for treating the virus in question in the future, or for identifying a compound to use in treating a different virus (including mutations or variants of the virus in question) in the future.

Without further elaboration, it is believed that one skilled in the art can use the preceding description to utilize the claimed inventions to their fullest extent. The examples and aspects disclosed herein are to be construed as merely illustrative and not a limitation of the scope of the present disclosure in any way. It will be apparent to those having skill in the art that changes may be made to the details of the above-described examples without departing from the underlying principles discussed. In other words, various modifications and improvements of the examples specifically disclosed in the description above are within the scope of the appended claims. For instance, any suitable combination of features of the various examples described is contemplated.

The invention is claimed as follows:

1. A method, comprising:
collecting chemical data including:
simplified molecular-input line-entry system (SMILES) representations of compounds;
viral protein amino acid sequences; and
compound-viral protein activities;
transforming bioactivities of the chemical data into pChEMBL values;
generating a plurality of regression models according to a plurality of different machine learning architectures;
generating a plurality of predicted activities for each pairing of one of the compounds and one of the viral protein amino acid sequences;
taking a consensus from a predefined number of top performing regression models selected from the plurality of regression models; and
outputting a list of the compounds according to the consensus having a highest predicted activity against the viral protein amino acid sequences.

2. The method of claim 1, wherein the SMILES representations of compounds are collected from MOSES and ChEMBL databases.

3. The method according to claim 1, wherein the viral protein amino acid sequences are collected from a Uniprot database.

4. The method according to claim 1, wherein the compound-viral protein activities are collected from NCBI, PubChem, and ChEMBL databases.

5. The method according to claim 1, wherein the plurality of different machine learning architectures include a Generalized Linear Model (GLM), Random Forests (RF), XGBoost, Support Vector Machines (SVM), Convolutional neural network (CNN), Long Short Term Memory (LSTM), CNN-LSTM, and Graph Attention Network (GAT)-CNN.

6. The method according to claim 1, wherein the top performing regression models are determined from the plurality of regression models based on performance with respect evaluation metrics including at least one of:
a mean absolute error;
a mean squared error;
a Pearson correlation R; and
a coefficient of determination.

7. The method of claim 1, wherein the predefined number of top performing regression models is five.

8. The method of claim 1, further comprising:
selecting a certain compound from the list having a lowest binding energy for at least one of the viral protein amino acid sequences examined; and
administering a therapeutically effective dose of the certain compound to a human to treat a virus associated with the viral protein amino acid sequences examined.

9. The method of claim 8, wherein the virus is SARS-COV-2 and the certain compound is Rifabutin.

10. A method of treating SARS-COV-2 in a human, comprising:
administering a therapeutically effective dose of Rifabutin.

11. The method of claim 10, wherein the therapeutically effective dose of Rifabutin is selected from a plurality of compounds for administration to the human from a plurality of candidate compounds at a plurality of dosages that includes Rifabutin at the therapeutically effective dose, wherein a consensus framework of a plurality of machine learning models evaluated each candidate compound of the plurality of compounds against spike proteins of SARS-COV-2, wherein Rifabutin at the therapeutically effective dose exhibits a highest compound-viral protein activity against the spike proteins from the plurality of candidate compounds according to the consensus framework.

12. A method of treating a virus in a human, comprising:
identifying viral protein amino acid sequences associated with the virus;
collecting chemical data including: simplified molecular-input line-entry system (SMILES) representations of a plurality of compounds and viral protein representations for the viral protein amino acid sequences; and
estimating compound-viral protein activities between each compound of the plurality of compounds and the viral protein amino acid sequences according to a plurality of machine learning models based on a regression task taking the SMILES representations and viral protein representations;
identifying a certain compound of the plurality of compounds having an estimated compound-viral protein activity against the viral protein amino acid sequences according to a consensus framework of the plurality of machine learning models that is above a threshold; and
administering a therapeutically effective dose of the certain compound to the human.

13. The method of claim 12, wherein the virus is SARS-COV-2 and the certain compound is Rifabutin.

14. The method of claim 12, wherein the simplified molecular-input line-entry system (SMILES) representations of a plurality of compounds and viral protein representations for the viral protein amino acid sequences are two-dimensional models.

15. The method of claim 12, wherein the plurality of machine learning models are developed via a corresponding plurality of different machine learning architectures including:
a Generalized Linear Model (GLM);
Random Forests (RF), XGBoost;
Support Vector Machines (SVM);
Convolutional neural network (CNN);
Long Short Term Memory (LSTM);
CNN-LSTM; and
Graph Attention Network (GAT)-CNN.
